(19) United States
(12) Patent Application Publication
(10) Pub. No.: US 2004/0001874 A1
(43) Pub. Date: Jan. 1, 2004

(54) Safe and effective nutritional supplement formulations and associated regimens adapted to prevent and/or treat targeted diseases or medical or health conditions, and related methods

(75) Inventors: Michael H. Davidson, Highland Park, IL (US); Bradley D. Edson, Scottsdale, AZ (US); Leslie D. Michelson, Beverly Hills, CA (US); Kenneth F. Lind, Scottsdale, AZ (US); Brian C. Smith, San Marino, CA (US); Randall E. Anderson, Phoenix, AZ (US)

Correspondence Address: HOGAN & HARTSON LLP, IP GROUP, COLUMBIA SQUARE, 555 THIRTEENTH STREET, N.W., WASHINGTON, DC, 20004, US

(73) Assignee: Vital Living, Inc.

(21) Appl. No.: 10/338,035

(22) Filed: Jan. 8, 2003

Related U.S. Application Data
(60) Provisional application No. 60/390,447, filed on Jun. 24, 2002.

(51) Int. Cl. A61K 47/00

(52) U.S. Cl. 424/439

(57) ABSTRACT

Disclosed herein are methods for identifying and developing safe and effective nutritional supplement formulations; associated regimens for administering such formulations; and methods for distributing such formulations. The nutritional supplement formulations and associated regimens are adapted to prevent and/or treat specific medical or health conditions. Also disclosed herein are methods for prescribing and supplying nutritional supplement formulations to patients according to appropriate regimens. Particular embodiments of the invention relate to safe and effective nutritional supplement formulations and associated regimens for the prevention and/or treatment of cardiovascular disease (“CVD”). Various embodiments of the invention provide advantages over the prior art in areas such as patient safety, medical effectiveness, physician supervision, patient compliance, and consumer costs.

### CROSS REFERENCE TO RELATED APPLICATIONS

This application claims benefit of priority from the filing date of U.S. provisional patent application Ser. No. 60/390,447, filed Jun. 24, 2002.

### FIELD OF THE INVENTION

The present invention relates to methods for identifying and developing safe and effective nutritional supplement formulations; associated regimens for the administration of such formulations; and distribution methods for such formulations. The formulations and associated regimens are adapted to prevent and/or treat specific diseases or other medical or health conditions by, among other things, providing targeted nutritional supplementation with improved patient compliance. Particular preferred embodiments of the invention relate to distribution methods and to safe and effective nutritional supplement formulations and associated regimens for the prevention and/or treatment of cardiovascular disease.

### BACKGROUND OF THE INVENTION

An expanding body of medical evidence shows the effectiveness of nutritional supplements in promoting health and suggests that nutritional supplements could be highly beneficial if successfully integrated into the provision of health care. American consumers spend billions of dollars per year on nutritional supplements, including vitamins, minerals, and herbs. One source estimates that 70% of adults use nutritional supplements, with over 30% of adults describing themselves as regular nutritional supplement users. A recent survey demonstrated that 63% of those surveyed wished they had more knowledge regarding how to use nutritional supplements to their benefit. In short, consumers are interested in and acknowledge the potential health benefits of proper nutritional supplementation.

Unfortunately, much of the consumption of nutritional supplements occurs without the specific recommendation or direction of a physician (i.e., the consumption of nutritional supplements by consumers is often “self-directed”). Many consumers research, select, and take nutritional supplements without physician guidance hoping to improve their general health, increase their longevity, and enhance their overall quality of life. Such self-directed use of supplements by consumers may be undesirable. First, consumers generally lack the ability to select safe and effective dosages and combinations of nutritional supplements without physician direction and supervision. Consumers that self-direct their nutritional supplementation may take supplement dosages or combinations that are ineffective or possibly even harmful. Second, since certain surveys estimate that approximately 18% of those taking prescription drugs also concurrently take nutritional supplements. This creates a risk that a nutritional supplement may be contra-indicated with a prescription drug. Thus, there is a need to introduce physician direction and supervision into the process of consumers' selecting and taking nutritional supplements.

The medical community is aware that carefully chosen and controlled nutritional supplementation may have potential benefits in the prevention and/or treatment of certain diseases, and medical or health conditions. Certain nutritional supplements, when properly administered, have been shown clinically to have a positive impact upon patient health. For example, glucosamine has been shown in clinical studies to promote joint health, saw palmetto has been shown in clinical studies to enhance prostate health, B vitamins have been shown in clinical studies to lower elevated homocysteine, and plant sterols have been shown in clinical studies to lower blood cholesterol. Furthermore, the medical community is aware that diseases such as cardiovascular disease (“CVD”), cancer, diabetes, and arthritis afflict a significant percentage of the general population. Merely by way of example, CVD negatively affects the health of over 60 million Americans and results in approximately one million deaths a year. Given the current accumulation of clinical evidence, the medical community has begun to focus on the fact that the prevention and/or treatment of many diseases could benefit from the introduction of safe and effective nutritional supplement formulations and associated regimens targeted at a specific disease or medical or health condition. The medical community similarly recognizes that there are significant portions of the general population, such as the obese, the elderly, women experiencing menopause, diabetics, cancer patients, those with chronic vascular, pulmonary or cardiac disease, and those with genetic anomalies who have particular medical or health needs which could be addressed by targeted nutritional supplementation. Unfortunately, the current health care environment lacks mechanisms for identifying, administering and supplying safe and effective nutritional supplements specifically targeted to particular diseases or medical or health issues.

Additional factors hinder the widespread acceptance and adoption of nutritional supplements as an integral part of physician-recommended treatments and/or prevention efforts. Nutritional supplements are subject to only minimal government regulation. Also, questions remain regarding the clinical substantiation of many popular nutritional supplements. Some nutritional supplements have been advertised with marketing claims that may not be substantiated by clinical evidence. Further, certain nutritional supplements may be manufactured from raw materials of varying quality. Certain supplements have been shown to contain contaminants that are potentially dangerous. Thus, some members of the medical community have reservations regarding the safety and effectiveness of nutritional supplements, and some physicians, therefore, may remain wary of recommending use of even clinically proven nutritional supplements.

For the above reasons, among others, nutritional supplements are often inefficiently or incorrectly used; they are also often not used enough (they are under utilized) when they could provide medical or general health benefits. The potential medical and health benefits of proper nutritional supplementation, therefore, remain largely unrealized. For example, although preventing and treating CVD is a top priority of modern health care, safe and effective nutritional supplementation nonetheless is largely underutilized to prevent and/or treat CVD. This is also the case with many other common diseases and medical or health conditions.

Thus, there remains a need in the art for improved methods to identify, supply and administer nutritional supplement formulations and regimens that enable safe and effective nutritional supplementation to prevent and/or treat various diseases and medical or health conditions.

### SUMMARY OF THE INVENTION

In view of the foregoing and other unmet needs, it is an object of the present invention to provide efficient methods for identifying safe and effective formulations of nutritional supplement ingredients for the prevention and/or treatment of certain diseases and medical or health conditions.

Similarly, it is an object of the present invention to provide a mechanism for identifying clinically safe and effective nutritional supplement formulations and associated regimens that are readily able to be prescribed by physicians and utilized by patients in compliance with a prescribed regimen. Such nutritional supplement formulations and their associated regimens include physician-directed nutritional supplementation programs that may complement prescribed pharmaceuticals.

Also, it is an object of the present invention to provide methods whereby safe and effective nutritional supplement formulations can be delivered to patients (with the participation of physicians) as products termed “nutraceuticals” or “nutraceutical formulations.” Such nutraceuticals are particularly targeted to advance the treatment and/or prevention of various common diseases or medical or health conditions.

Additionally, it is an object of the present invention to provide regimens for administering and supplying nutraceutical formulations to patients in order to advance the treatment and/or prevention of various common diseases or medical or health conditions.

Furthermore, it is an object of the present invention to provide nutraceuticals that employ targeted formulations of nutritional supplement ingredients and associated administering regimens that are specifically adapted to prevent and/or treat one or more specific medical or health conditions without interfering with common pharmaceutical drugs.

Concurrently, it is an object of the present invention to identify nutritional supplement formulations that are safe and effective for a large portion of patients generally regardless of the various diseases or medical or health conditions they may possess or the medications they may be taking without the need for close monitoring to ensure safety.

It is also an object of the present invention to provide nutraceuticals comprising formulations, administering regimens, and compliance programs that are particularly adapted for the prevention and/or treatment of CVD.

To achieve these and other objects, nutritional supplement formulations and regimens according to the invention are adapted to reduce risk factors for specific diseases, or medical or health conditions and thus assist in the primary and secondary prevention of adverse medical events associated with those diseases or conditions. The formulations and associated regimens according to the invention are also adapted to treat patients suffering from disease or having various medical or health conditions.

Embodiments according to a first aspect of the present invention comprise methods for identifying and developing nutritional supplement formulations and regimens. Such methods generally survey the scientific literature to identify reliable studies pertaining to the effect of various nutritional supplement ingredients in preventing and/or treating a medical or health condition of interest, and identify desired nutritional supplement ingredients, formulations and dosages from those surveyed studies. The desired ingredients may then be combined to produce a safe and effective nutritional supplement formulation and regimen for administering the formulation to treat and/or prevent a given disease or medical or health condition. Nutritional supplement ingredients are generally not included in a given nutritional supplement formulation according to the present invention, unless clinically proven evidence demonstrating the ingredient's beneficial impact on the reduction of risk factors or occurrence of negative events for specific diseases or medical or health conditions is available. Alternatively or in addition, national guidelines recommending use of the nutritional supplement ingredients to address the particular diseases or medical or health conditions may also be available to support inclusion of an ingredient in a given nutritional supplement formulation.

Additionally, the methods for developing nutritional supplement formulations and regimens according to preferred embodiments of this first aspect of the present invention select and combine nutritional supplement ingredients into the nutritional supplement formulations where those ingredients have individual beneficial medical or health impacts that are substantially additive to one another. These preferred embodiments of this first aspect of the present invention enable the development of such substantially additive formulations by identifying and selecting for combination those nutritional supplement ingredients which are effective, which are safe for use (alone and with each other), and which operate through substantially independent mechanisms of action in the body.

According to embodiments of a second aspect of the present invention, a method for prescribing and supplying nutritional supplement formulations to patients according to appropriate regimens is provided. Embodiments of this second aspect of the present invention can resemble a prescription drug process in their implementation. According to embodiments of this second aspect of the invention, physicians select appropriate nutritional supplement formulations and give instructions to patients as to how to take those formulations; the patients then obtain the nutritional supplement formulation and follow the instructions provided by the physician.

In preferred embodiments of this second aspect of the present invention, patients will be encouraged to begin an appropriate nutraceutical regimen (e.g., a course of particular nutraceutical dosages and methods of administering and supplying same) following their discharge or initial visit with the directing physician. Such regimens can include charging the patient a one-time fee for their first month's supply of nutraceutical doses, or alternatively giving an initial few weeks worth of free nutraceutical from the physician, to get the patient started on the directed regimen immediately and concurrently having the patient join a nutraceutical delivery program. By enrolling, the patients authorize a distributor to deliver the appropriate nutraceutical supply, by mail for example, on a periodic basis, such as monthly.

In certain of the above preferred embodiments according to the second aspect of the invention, where the directing physician gives the patient an initial supply of nutraceutical doses, this initial supply can optionally be provided in a pre-packaged lifestyle and compliance program kit for physician distribution to the patients.

A third aspect of the invention includes nutritional supplement formulations and regimens that are safe and effective for the prevention and/or treatment of CVD. Embodiments of such formulations and regimens according to this third aspect of the invention satisfy the criteria for developing safe and effective formulations and regimens associated with the first aspect of the invention, and also can be prescribed and supplied in accordance with the second aspect of the invention. Embodiments of this third aspect of the invention include a formulation that comprises various nutritional supplement ingredients included in safe and effective amounts. Nutritional supplement ingredients included in safe and effective dosages within such formulations include folic acid, vitamin B6, vitamin B12, niacin, plant sterols (such as beta sistosterol, campesterol, stigmasterol, and brassicasterol), and fish oil (containing omega-3 fatty acids such as EPA and DHA).

In preferred embodiments of this third aspect of the present invention, the nutritional supplement formulation is provided in a nutraceutical product. Such nutraceuticals may be delivered to patients in single-serving packets, the contents of which are taken twice daily by the patient. The contents of such packets preferably include multivitamin pills having a functional dosage of folic acid, vitamin B9, and vitamin B12, niacin tablets, plant sterol softgels, and plant sterol softgels. Supplying patients with nutraceutical packets and directing the patients to, for example, take the contents of a single packet twice per day makes it easier for patients to comply with the directed dosage regimens.

Various preferred aspects and embodiments of the invention will now be described in detail with reference to figures.

### BRIEF DESCRIPTION OF THE DRAWINGS

FIG. 1 is a flow diagram depicting a method for identifying and developing nutritional supplement formulations and regimens according to preferred embodiments of the first aspect of the present invention.

FIG. 2 is a flow diagram depicting a distribution method for prescribing and supplying nutraceuticals to patients according to preferred embodiments of the second aspect of the present invention.

### DETAILED DESCRIPTION OF THE PREFERRED EMBODIMENTS

A first aspect of the present invention includes methods for identifying and developing safe and effective nutritional supplement formulations and associated regimens for administering and supplying such formulations to patients. The formulations and regimens according to the invention are adapted to reduce risk factors for specific diseases, or medical or health conditions and thus assist in the primary and secondary prevention of adverse medical events associated with those diseases or conditions. The formulations and regimens according to the invention are also adapted to treat patients suffering from disease or having various medical or health conditions.

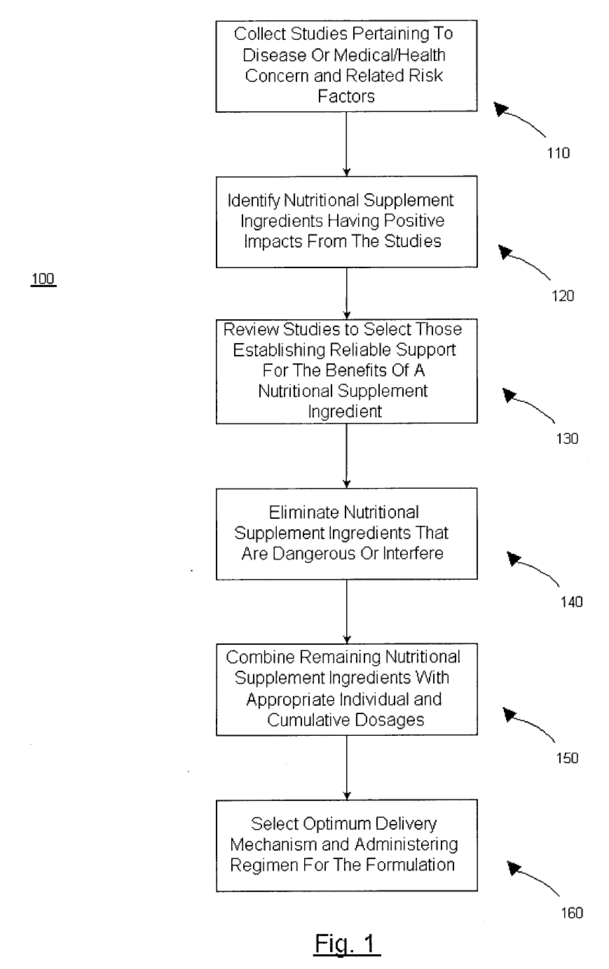

Referring to FIG. 1, there is depicted one method for identifying and developing nutritional supplement formulations and regimens according to preferred embodiments of the first aspect of the present invention. As depicted, a preferred identifying and developing method 100 according to the first aspect of the invention comprises initially surveying the scientific literature and to collect 110 studies pertaining to the disease or health or medical condition of interest, or pertaining to known risk factors for the disease or health or medical condition. Once the appropriate literature and studies have been collected at step 110, they are then reviewed to identify 120 studies claiming to demonstrate or otherwise alleging a positive effect or impact of a nutritional supplement ingredient upon the disease or medical or health condition of interest.

These identified studies are then reviewed to select 130 those studies that establish a reliable positive effect or impact of a particular nutritional supplement ingredient. In these embodiments of this first aspect of the invention, the studies are generally not considered reliable enough to establish a positive effect or impact from a given nutritional supplement ingredient unless supporting evidence is available that clinically demonstrates the ingredient's beneficial impact on the reduction of risk factors for specific diseases or medical or health conditions. Alternatively or in addition, a nutritional supplement ingredient can be deemed to have reliable positive effect or impact at step 130 if there are national guidelines recommending use of the nutritional supplement ingredient to address the particular diseases or medical or health conditions.

After step 130, a subset of the original studies collected at step 110 remain. Next, the nutritional supplement ingredients that have been shown to have positive clinical impacts are each reviewed individually and collectively in detail at step 140 to eliminate any supplement ingredients that are potentially dangerous or could interfere with other drugs, treatments, etc., for a particular patient. For example, various nutritional supplements selected at step 130 as being demonstrated to have a positive impact on a particular risk factor may also be known to interfere with a particular prescription drug commonly taken by patients suffering from the disease of interest. In this case, this nutritional supplement ingredient would be eliminated 140 from consideration for inclusion into a nutraceutical formulation according to the present invention. Similar eliminations would occur if a nutritional supplement ingredient was found to be toxic or have significant side effects.

Once nutritional supplement ingredients have been selected, and any dangerous or interfering ingredients are eliminated, the remaining nutritional supplement ingredients are combined 150 in appropriate individual and/or cumulative dosages. The individual dosages are preferably identified from the surveyed studies as being those dosages which produce a safe and effective impact to treat and/or prevent a given medical or health condition.

According to most preferred embodiments of this first aspect of the present invention, the choices regarding nutritional supplement ingredients included at step 130 or eliminated at step 140 in the nutritional supplement formulations, and the choices regarding the relative dosages selected at step 150, are made with a goal of producing a resulting nutritional supplement formulation that contains nutritional supplement ingredients whose individual impacts are substantially additive. For example, assume ingredient W provides a +10 benefit for factor F1, component X provides a +5 benefit for factor F1 and a +15 benefit for factor F2, component Y provides a +10 benefit for factor F2, and component Z provides a +5 impact for F1 and a +20 impact for F3. A completely “additive” formulation of these three ingredients would provide a +20 benefit for factor F1, a +25 benefit for factor F2, and a +20 benefit for factor F3 without requiring an increase in dosage of any of the four component ingredients. Due to the interactions of nutritional supplements and pharmaceuticals in a patient's body, however, it may be difficult to identify such beneficial additive formulations. Accordingly, the present invention creates such substantially additive formulations by identifying and selecting for combination not only those nutritional supplement ingredients which are effective and which are safe for use (alone and with each other), but also those ingredients that operate through substantially independent mechanisms of action in the body. In this manner, the individual nutritional supplement ingredients are unlikely to interfere with one another in the body and thus produce a maximum positive result.

Referring back to FIG. 1, the preferred identifying and developing method 100 according to the first aspect of the invention lastly selects 160 an appropriate delivery mechanism and administering regimen for the finalized formulation and dosages produced at step 150. For example, various supplement delivery mechanisms can be employed for the formulation, including single tablets, capsules, softgel capsules, and the like, as well as edible bars, wafers, and powders. Additionally, one or more of the above can be combined (multiple pills, capsules and/or edible bars, etc.) in single-use packaging depending on a particular formulation. Additionally, administering regimens can be varied along with the delivery mechanism to achieve a balance of patient usage convenience with logistical concerns such as price impacts and formulation issues.

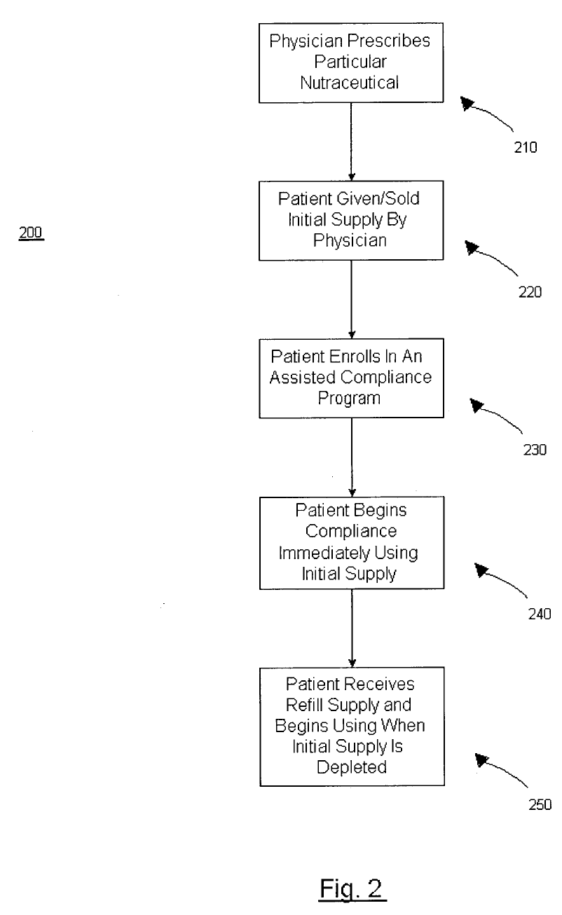

Referring now to FIG. 2, there is depicted a distribution method 200 for prescribing and supplying nutraceuticals to patients according to preferred embodiments of the second aspect of the present invention. As shown in FIG. 2, a preferred distribution method 200 for prescribing and supplying nutritional supplement formulations (or nutraceutical products) according to appropriate administering regimens can resemble that of the prescription drug process. More specifically, physicians first prescribe an appropriate nutritional supplement treatment 210 by selecting appropriate nutritional supplement formulations (or nutraceutical products) and giving instructions to patients as to how to take those formulations (or products). The patients are then left to follow the physician's prescribing instructions by obtaining and/or taking the supplements according to the regimen. In certain of such embodiments, following discharge or an in-office visit with a physician, patients will be encouraged to begin an appropriate nutraceutical regimen (e.g., a course of particular nutritional supplement dosages and methods of administering and supplying same) according to the present invention as described in more detail below.

Following the prescribing by the physician, a patient can be charged a one-time fee for their first month's supply of nutraceutical doses or they can be given a few weeks worth of free nutraceutical doses by the physician at step 220 such that the patient is encouraged to start on the directed regimen immediately (while they proceed to join a nutraceutical delivery program as described below). Step 220 of this preferred embodiment, while not essential, is beneficial because it helps ensure initial patient compliance by enabling the patient to start following the nutraceutical regimen immediately after receiving the instructions from the physician without having to travel first to a store to locate and purchase the appropriate nutraceutical. Additionally, having physicians stock small quantities of nutraceuticals, in a manner similar to prescription drug samples, has the added benefit of helping physicians become familiar with the nutraceuticals and thus become more comfortable directing patients to use them.

In embodiments of this second aspect of the invention where the directing physician gives or sells the patient an initial supply of nutraceutical doses at step 220, this initial supply can optionally be provided in a pre-packaged lifestyle and compliance program kit for physician distribution to the patients. This kit provides materials (along with the prescribed nutraceutical) designed to help make certain that patients follow through with their physician-directed nutraceutical regimen. Such a kit could include, for example, an instructional video or booklet and a chart or checklist type lifestyle program tracker. Alternatively, the instructional and program tracker materials could be integrated into a simple-to-use computer program.

Referring again to FIG. 2, the patient thereafter preferably enrolls 230 (either on their own or through the physician) in an assisted compliance program. By enrolling, the patient authorizes a distributor to deliver the appropriate nutraceutical supply, by mail for example, on a periodic basis, such as monthly. This is a convenient way for the patient to continue the directed nutraceutical regimen without having to make repeat trips to the drug store or vitamin retailer, and, therefore, lessens the potential of the patient falling out of compliance by simply failing to proactively refill their nutraceutical supply. Such a monthly shipment may be beneficially employed to deliver compliance program collateral materials, thus reinforcing the program's many benefits. This mail-order approach is also advantageous because the level of quality control and physician direction provided approaches that of prescription pharmaceutical regimens in that the patient has reasonable assurances that he or she is consuming a product that is of a certain quality, efficacy and safety. Also, this is advantageous because the physician does not have to stock inventory (other than the start-up kits).

Upon leaving the prescribing visit with the physician, the patient begins compliance 240 with the prescribed regimen immediately by beginning to take the initial physician supplied doses of the nutraceutical as directed. Later, at step 250, the patient receives the first of his or her refill supplies provided by enrollment in the assisted compliance program, such as via mail as described above, and will begin to take doses from the refill shipment once the initial supply is depleted.

Embodiments of a third aspect of the present invention comprise nutritional supplement formulations and associated regimens for the prevention and/or treatment of CVD. These CVD-specific nutraceuticals combine safe and effective nutritional supplement ingredients that beneficially impact upon CVD and cardiovascular health generally in an additive manner. As described in detail below, this aspect of the invention provides targeted cardiovascular nutritional supplementation that includes appropriate vitamins and minerals, therapeutic dosages of folate and niacin, plant sterols, and omega-3 fatty acids. The CVD-specific nutraceuticals as disclosed herein support the specific nutritional needs of cardiovascular patients by improving blood lipids and cholesterol levels, as well as by providing nutrients proven to promote cardiovascular health.

Various known risk factors are linked with poor cardiovascular health. Cholesterol is probably the most well known. In particular, higher LDL cholesterol (the “bad” cholesterol) levels are linked clinically with increased probability of CVD while higher HDL cholesterol (the “good” cholesterol) levels have recently been linked with decreased probability of CVD. Additionally, high blood levels of homocysteine, an amino acid, have been found to be a significant risk factor for CVD. Similarly, elevated triglyceride (linked to saturated fat intake) levels, and particularly in association with elevated LDL cholesterol levels, has been correlated with the development of atherosclerosis, the underlying cause of heart disease and stroke. Furthermore, high sensitivity C-reactive protein (“hs-CRP”) is a known inflammatory marker for increased CVD risk. Therefore, the nutritional supplement formulations and regimens for the prevention and/or treatment of CVD according to embodiments of this aspect of the invention were developed with the simultaneous goals of reducing LDL cholesterol levels, reducing total triglyceride levels, increasing HDL cholesterol levels, decreasing homocysteine levels, and decreasing hs-CRP levels.

Preferred embodiments of this aspect of the present invention employ the combination therapy of folic acid (vitamin B-9), cyanocobalamin (vitamin B-12) and pyridoxine hydrochloride (vitamin B-6) that has been associated with a reduction in plasma homocysteine levels. Further, such embodiments employ plant sterol therapy to lower blood cholesterol levels by interfering with the absorption of fats, and omega-3 fatty acids therapy to reduce blood triglycerides. Additionally, such preferred embodiments include niacin which has been shown to raise beneficial HDL cholesterol levels while lowering triglycerides and modestly reducing LDL cholesterol levels.

The nutraceutical regimen according to this preferred embodiment of the invention entails the consumption of the contents of one pill packet at two different times each day. Each packet comprises CVD-specific multivitamin pills, niacin tablets, plant sterol softgels and fish oil softgels necessary to provide a therapeutically effective amount of the selected nutritional supplement ingredients, such as, for example, in the manner as shown in Table 1 below.

TABLE 1

| QTY | PILLS |
|---|---|
| 1 | Multivitamin with functional |
| | dosage of Folic Acid, B6, B12 |
| 1 | Niacin |
| 3 | Plant Sterol Softgel |
| 1 | Fish Oil Softgel |

Plant steropls interfere with the absorption of fat by the body. Therefore, patients are directed to ingest the contents of a first packet (containing half of a dail dosage of all nutritional supplement ingredients, including the plant sterols) before their first meal of the day that contains fat. The patients are also directed to take a second packet (containing the second half of the daily dosage) later in the day just before dinner (because dinner normally contains fat). By directing the patients to take the contents of a packet at two separate times according to this preferred administering regimen, the interference effect plant sterols have on the absorption of fat is effectively spread across multiple meals. Furthermore, having the patient take two half-doses of nacin as opposed to a single full dose has the additional benefical impact in that it helps minimize the potential for patients to experience a niacin flush.

Further, providing patients with a supply of identical packets (as indicated above with respect to Table 1) and directing the patients to take the contents of a single paket twice per day makes it easier for patients to comply with the directed dosages. This greatly increases the chances that the patient will continue in compliance with the directed regimen long enough to obtain significant health benefits. Additionally, the pre-packaging of various, proven safe and effective nutritional supplement formulations into ready-to-take packets takes advantage of economies of scale. Thus, patients are provided with a product that is superior in terms of safety, effectiveness, and ease of use that nonetheless costs less than if the patients had tried to purchase the ingredients individually. Moreover, the compilation of various safe and effective nutritional supplement formulations into a nutraceutical targeted to a single medical or health condition, as exemplified by the particular CVD-specific nutraceutical disclosed herein, may eliminate the need for patients to take general multivitamins or other supplements.

Notably, certain popular products such as garlic, high-dose vitamin E, or isoflavones are currently excluded from the CVD-specific nutraceutical according to most preferred embodiments of this aspect of the present invention because adequate scientific support is not currently available to justify their inclusion at this time. In compliance with the criteria utilized in the identifying and developing method aspects of the invention as described above, the nutritional supplement ingredients included within the particular CVD-specific nutraceutical according to the present invention satisfy at least one of the following three criteria: 1) proven clinical trial evidence demonstrating the beneficial effects on cardiovascular risk factors; 2) clinically proven evidence to reduce cardiovascular events (myocardial infarction, total CVD, or strokes); or 3) national guidelines recommending the nutritional supplement to reduce the risk of CVD.

Furthermore, tablets, softgel capsules, and the like, as described above in Table 1, are a preferred mechanism to deliver the CVD-specific nutritional supplement formulation as described herein because, when aggregated in dosage packets as described above, they are easier for patients to integrate into their lifestyle. Alternatively, of course, other suitable delivery forms can be used to provide an appropriate amount of a given nutritional supplement to a patient, including bars, wafers, and powders. Preferably, the individual nutritional supplement ingredients are of the highest quality, including odorless omega-3 oil and the highest potency plant sterols.

Applicants have found that the particular types and amounts of nutritional supplement ingredients employed in the CVD-specific nutraceutical disclosed herein complement one another as well as common pharmaceuticals. More specifically, as shown in Table 1, the packets used in the CVD-specific nutraceutical according to this preferred embodiment of present invention each contain a single multivitamin tablet. The particular multivitamin, taken twice a day as directed, provides delivery of the various vitamins and minerals as depicted in Table 2 below in terms of daily dosage and corresponding percent of recommended daily intake (“RDI”).

TABLE 2

| VITAMINS AND MINERALS | mg | RDI % |
|---|---|---|
| Iodine (Potassium Iodide) | 0.15 | 100% |
| Chromium Picolinate | 0.12 | 100% |
| Selenium (AAC) | 0.07 | 100% |
| Zinc (AAC) | 15.00 | 100% |
| Copper (AAC) | 2.00 | 100% |
| Magnesium (AAC) | 100.00 | 25% |
| Mangnese (AAC) | 2.00 | 100% |
| Molybdinum (AAC) | 0.08 | 100% |
| Calcium (AAC) | 100.00 | 10% |
| Vitamin B-1 (Thiamine | 1.50 | 100% |
| nitrate) | | |
| Phosphorous | 96.00 | 10% |
| Vitamin B-2 (Riboflavin) | 1.70 | 100% |
| Vitamin B-5 (Ca | 10.00 | 100% |
| Pantothenate) | | |
| Vitamin C | 60.00 | 100% |
| Vitamin D3 (100,000 IU/g) | 400 IU | 100% |
| Vita-E (Acetate 950 IU/g) | 30 IU | 100% |
| d-Biotin (1%) | 0.30 | 100% |
| Potassium | 160.00 | 4% |
| Chloride | 144.00 | 4% |
| Vitamin B-6 (Pyridoxine | 25.00 | 1250% |
| HCL) | | |
| Vitamin B-9 (Folic Acid) | 0.80 | 200% |
| Vitamin B-12 | 1.00 | 16667% |
| (Cyanocobalamin) | | |

Studies have demonstrated that many patients with CVD do not achieve the RDI for various vitamins and minerals. It may not be advisable, however, for a CVD patient to take a standard multivitamin because such a vitamin may provide either too little or too much of a specific nutritional supplement ingredient. For example, most multivitamin formulas contain vitamin K which is believed to possibly interfere with warfarin, a prescription pharmaceutical often given to cardiac patients (commonly known as the branded drug Coumadin®). Further, iron, an oxidizing agent, is also typically present in standard multivitamins. Several studies have preliminarily indicated that the regular intake of iron via a multivitamin may enhance the oxidation of LDL cholesterol and thus promote the development of atherosclerosis. In addition, many multivitamins include dosages of vitamin E or other anti-oxidants that have not been proven to reduce the risk of heart disease. The particular multivitamin composition depicted in Table 2 above and according to the invention, takes into account these factors to create a multivitamin component to the nutraceutical specifically adapted for safe and effective use in CVD prevention and/or treatment. Additionally, this nutraceutical most preferably provides essential RDI nutrients in highly absorbable forms.

Furthermore, as shown above in Table 2, nutrients such as folic acid and vitamins B6 and B12, which are often not present in a standard multivitamin in effective amounts, are purposely included in the multivitamin composition of this preferred embodiment of this aspect of the present invention. Folic acid (present in approximately 800 mcg amount) and vitamins B6 (approximately 25 mg) and B12 (approximately 1 mg) all play a role in the metabolism of homocysteine, an amino acid in which high levels in the blood are a risk factor for CVD. These vitamins lower homocysteine levels and have been shown to reduce endothelial dysfunction, an early marker of atherosclerosis. In addition, there is evidence that these homocysteine-reducing vitamins reduce the rate of angioplasty restenosis and the progression of atherosclerosis. Therefore, the inclusion of folic acid and the B vitamins in the approximate dosages depicted above provide benefits directed to the prevention and/or treatment of CVD that would not be provided by a standard multivitamin.

The niacin tablets contained in each packet as depicted in Table 1, each preferably provide 250 mg of niacin, thus providing a cumulative niacin daily intake of 500 mg. Niacin intake is a proven therapy to raise HDL cholesterol and lower triglycerides at low doses (500-1000 mg/day) and decrease LDL cholesterol modestly at higher doses (1000-3000 mg/day). The most common side effect associated with niacin intake is flushing, which can be controlled by taking the niacin with food. Niacin is included in the CVD-specific nutraceutical formulation as disclosed herein because of its proven benefits in modifying lipids and due to its potential in reducing atherosclerotic development if used in conjunction with a statin. Furthermore, niacin independently has been demonstrated to reduce cardiovascular morbidity and mortality.

Optionally, the dosage of niacin contained in the initial month starter supply of nutraceutical which may be provided by the physician, is lower than the amount included in subsequent packets of nutraceutical purchased by and/or delivered to the patient. Such an initial lower dosage could help to reduce niacin flush in patients as they start the nutraceutical regimen and slowly build a tolerance to the nutrient before the full dosage (contained in the later packets) is begun. Also optionally, the niacin dosage in each packet could be provided in two pills (instead of one as indicated in Table 1). This way, a patient can be instructed to take only one of the two niacin pills in each packet to avoid niacin flush symptoms until a tolerance is developed.

The plant sterols softgel capsules (3 capsules per packet for a total of six per day) indicated above in Table 1 provide approximately 1.8 g (1800 mg) total daily of mixed sterols as indicated below in Table 3.

TABLE 3

| | Compound | Dosage | |
|---|---|---|---|
| Functional Nutrient | provided | (mg) | |
| Free Plant Sterols | Beta Sistosterol | ˜890 | mg |
| | Campesterol | ˜530 | mg |
| | Stigmasterol | ˜290 | mg |
| | Brassicasterol | ˜30 | mg |

| | | |
|---|---|---|
| Total Sterols | 1800 | mg |

The National Cholesterol Education Program (NCEP) Adult Treatment Panel (ATP) III has indicated that plant stanol/sterol esters could be used as a therapeutic option to enhance LDL cholesterol lowering in the range of 6-15%. The plant sterols softgels used in this preferred embodiment are included to provide such a therapeutic effect. As will be understood by one skilled in the art, the above total sterol dosage can be modified within therapeutically effective ranges without departing appreciably from this beneficial effect. Further, the exact relative amounts and types of plant sterols present in the softgel beta sistosterol, campesterol, stigmasterol, etc.) can also vary without substantially decreasing beneficial effects.

Notably, the multiple sterol capsules in each packet as shown in Table 1, instead of a single larger softgel, makes swallowing easier for the patient as well as accommodates common industry-sized softgels. The number of sterol softgel capsules, of course, can be modified as necessary to accommodate larger or smaller sized softgels.

The fish oil softgels described in Table 1 contain a mixture of omega-3 fatty acids with the 2 softgels per day providing 1100 mg total of EPA, DHA and other omega-3 fatty acids. Omega-3 fatty acids reduce the rate of deaths in patients with pre-existing CVD and have been found to lower triglycerides by 10-20% at doses of approximately 1100 mg daily. Table 4 below demonstrates the approximate dosages of relevant omega-3 fatty acids (total amounts may vary slightly according to exact fish oil used) present in the fish oil capsules indicated in Table 1.

TABLE 4

| | Compound | Dosage |
|---|---|---|
| Functional Nutrient | provided | (mg) |
| Fish Oil | EPA | ˜600 mg |
| (2 Capsules) | DHA | ˜400 mg |
| | Other Omega-3 | ˜100 mg |
| | Fatty Acids | |

| | |
|---|---|
| Total Omega-3 Fatty Acids | 1100 mg |

As is the case with the plant sterols, the above fish oil dosages can be modified within therapeutically effective ranges without departing appreciably from its intended beneficial effect. Further, the exact composition of the omega-3 fatty acids contained in the fish oil used will vary without substantially decreasing the realization of the intended beneficial effects.

In certain alternative embodiments of the CVD-specific nutraceutical herein disclosed, it is possible to blend co-enzyme Q10 (also known as ubiquinone) within the fish oil capsules to reduce the amount of fish oil needed by the patient in each packet. Co-enzyme Q10 is a potent antioxidant that is produced during the synthesis of cholesterol and which is known to be utilized in the body for normal muscle function. Statins, which inhibit the hepatic synthesis of cholesterol, may reduce plasma co-enzyme Q10 levels as a side effect. This statin-induced decrease in co-enzyme Q10 levels is considered one of the potential causes of muscle toxicity (myopathy) associated with statin use. The inclusion of co-enzyme Q10, such as in, for example, approximately 25-30 mg amounts, into each fish oil capsule can help reduce statin-induced myopathy and thus enhance the safety of and patient compliance with statin therapy.

The particular CVD-specific nutraceuticals and regimens herein disclosed were also designed to achieve a measurable benefit on lipid levels equivalent to those provided by many popular pharmaceutical drug therapies. The expected range of benefits (“NC” indicating no change) is projected as indicated below in Table 5.

TABLE 5

| ACTIVE | | | TRI- | HOMO- |
|---|---|---|---|---|
| COMPOUNDS | LDL | HDL | GLYCERIDES | CYSTEINE |
| Plant Sterols | decrease | increase | NC | NC |
| | 5-15% | 0-3% | | |
| Niacin | NC | increase | decrease | NC |
| | | 5-10% | 0-10% | |
| Omega-3 Fatty | NC | increase | decrease | NC |
| Acids | | 0-5% | 10-20% | |
| Folic Acid, | NC | NC | NC | decrease |
| Vitamins B6 | | | | 10-20% |
| and B12 | | | | |

Given that all of the nutritional supplement ingredients used in the CVD-specific nutraceutical herein disclosed operate according to independent mechanisms, the cumulative effective on LDL cholesterol, HDL cholesterol, triglycerides and homocysteine levels would be additive and would not counteract each other or interfere with common CVD prescription pharmaceutical treatments.

As described above, the particular CVD-specific nutraceutical according to this particular preferred embodiment of the invention additively combines only clinically proven nutritional ingredients for maximizing health in the following areas: LDL and HDL cholesterol, total triglycerides, homocysteine and C-reactive protein.

In sum, as will be readily apparent after reading the present disclosure, among other things, the present invention provides advantageous methods for identifying safe and effective formulations of nutritional supplement ingredients, along with related regimens for administering such formulations to patients, that advance the treatment and/or prevention of various common health issues, medical conditions, or diseases. Nutraceutical formulations and associated regimens should be targeted formulations and regimens that are specifically adapted to prevent and/or treat one or more specific diseases or medical or health conditions. Furthermore, the various embodiments of the invention as disclosed and described above also make it easier for patients to comply with the directed regimen and thus increase the chances that beneficial results will be achieved.

While preferred embodiments of the present invention have been shown and described herein, it will be obvious to those skilled in the art such embodiments are provided by way of example only. Numerous insubstantial variations, changes, and substitutions will now be apparent to those skilled in the art without departing from the scope of the invention disclosed herein by the Applicants. For example, the particular dosage amounts indicated in the various tables above relate solely to a preferred embodiment of the invention and one of ordinary skill in the art will readily appreciate that the amounts indicated may be varied within clinically safe and effective parameters without losing the beneficial aspects herein disclosed. Also, the particular regimens described herein relate solely to preferred embodiments of the invention and one of ordinary skill will readily appreciate that the regimens indicated may be varied within safe and effective parameters. Accordingly, it is intended that the invention be limited only by the spirit and scope by the claims as follows.

1. A method for identifying and developing safe and effective formulations of nutritional supplements targeted to treat and/or prevent a specific disease or medical or health condition, said method comprising:
collecting scientific literature relating to said disease or condition;
reviewing said literature to identify nutritional supplement ingredients having a positive effect in the treatment or prevention of said disease or condition or having a positive impact on a risk factor associated with said disease or condition;
selecting nutritional supplement ingredients from the identified nutritional supplement ingredients which meet predefined standards of safety and effectiveness;
combining said selected nutritional supplement ingredients in safe and effective dosages into a nutritional supplement formulation.

2. The method according to claim 1, wherein said identified nutritional supplement ingredients are selected and combined to produce a formulation that provides substantially additive positive impacts and effects from said combined dosages of said nutritional supplement ingredients.

3. The method according to claim 2, wherein said positive impacts and effects from nutritional supplement formulation ingredients are substantially additive because said selected and combined nutritional supplement ingredients operate physiologically through independent mechanisms of action.

4. The method according to claim 1, wherein said identified nutritional supplement ingredients meet predefined standards of safety and effectiveness if one or more items of said reviewed literature provide supporting evidence that clinically proves a given ingredient's beneficial impact or effect.

5. The method according to claim 5, wherein said identified nutritional supplement ingredients meet predefined standards of safety and effectiveness also if national guidelines exist that recommend the use of said given nutritional supplement ingredient to address said disease or condition.

6. The method according to claim 1, wherein said identified nutritional supplement ingredients meet predefined standards of safety and effectiveness if national guidelines exist that recommend the use of a given nutritional supplement ingredient to address said disease or condition.

7. The method according to claim 1, wherein said selecting step comprises eliminating any nutritional supplement ingredients that interfere with pharmaceutical drugs commonly prescribed to treat or prevent said disease or condition.

8. The method according to claim 1, wherein said selecting step comprises eliminating any nutritional supplement ingredients that would require careful monitoring to detect negative interactions or side effects if administered without direct supervision by a physician.

9. The method according to claim 1, further comprising designing an optimum delivery mechanism and optimum administering regimen for said formulation.

10. The method according to claim 9, wherein said designing step comprises selecting a delivery vehicle for said formulation from the group consisting of tablets, capsules, softgels, edible bars, edible wafers, and powders.

11. The method according to claim 9, wherein said optimum delivery mechanism comprises packaging said selected nutritional supplement ingredients together in single-use packages, wherein each package is adapted to deliver a single dose of said nutritional supplement formulation.

12. The method according to claim 11, wherein said nutritional supplement ingredients in said single use packages are contained in one or more delivery vehicles, said delivery vehicles being selected from the group consisting of tablets, capsules, softgels, edible bars, edible wafers, and powders.

13. The method according to claim 9, wherein said optimum administering regimen is adapted to balance patient usage ease and convenience with logistical concerns.

14. A method for physician directed nutritional supplementation targeted to treat and/or prevent a specific disease or medical or health condition in a patient, said method comprising:
diagnosing the patient as needing nutritional supplementation for said disease or condition;
prescribing a nutritional supplement formulation specifically adapted to prevent and/or treat said disease or condition, said nutritional supplement formulation containing safe and effective dosages of nutritional supplement ingredients;
giving said patient an initial supply of a nutraceutical product adapted to deliver said prescribed nutritional supplement formulation according to a physician-directed regimen, said patient being instructed to begin compliance with said regimen immediately by accordingly taking doses of said initial nutraceutical supply; and
enrolling said patient in an assisted compliance program adapted to facilitate distribution of refill supplies of said nutraceutical produced to said patient as necessary.

15. The method according to claim 14, wherein said prescribed nutritional supplement formulation comprises safe and effective dosages of nutritional supplement ingredients that either have a positive effect in the treatment or prevention of said disease or condition or have a positive impact on a risk factor associated with said disease or condition.

16. The method according to claim 14, wherein said prescribed nutritional supplement formulation combines various nutritional supplement ingredients that have substantially additive individual positive impacts and effects.

17. The method according to claim 16, wherein said positive impacts and effects from said nutritional supplement formulation ingredients are substantially additive because said nutritional supplement ingredients operate physiologically through independent mechanisms of action.

18. The method according to claim 14, wherein said prescribed nutritional supplement formulation does not contain any nutritional supplement ingredients that may interfere with pharmaceutical drugs commonly taken to treat or prevent said diagnosed disease or condition.

19. The method according to claim 14, wherein said nutraceutical product comprises single-use packages containing a dose of said nutritional supplement formulation.

20. The method according to claim 19, wherein said single-use packages contain said nutritional supplement ingredients for a dose of said formulation, said nutritional supplement ingredients together or separately provided in one or more delivery vehicles, said delivery vehicles being provided in delivery forms selected from the group consisting of tablets, capsules, softgels, edible bars, edible wafers, and powders.

21. The method according to claim 14, wherein said initial supply of said nutraceutical is given to the patient without charge by the prescribing doctor.

22. The method according to claim 14, wherein said initial supply of said nutraceutical is sold to the patient by the prescribing doctor.

23. The method according to claim 14, wherein said initial supply of said nutraceutical is pre-packaged in a compliance program kit, said kit containing said nutraceutical and materials adapted to ensure that the patient complies with said physician directed regimen.

24. The method according to claim 23, wherein said materials in said kit are selected from the group consisting of instructional videos, instructional books, instructional computer programs, compliance charts, compliance checklists, and compliance tracking computer programs.

25. The method according to claim 14, wherein said enrolling step is performed at the prescribing physician's office.

26. The method according to claim 14, wherein said assisted compliance program comprises pre-arranging for the delivery of said refill supplies of said nutraceutical to the patient.

27. The method according to claim 26, wherein supplemental informational or instructional materials are delivered with said refill supplies to the patient.

28. The method according to claim 26, wherein said nutritional supplement formulation of said nutraceutical in said initial supply is different from said nutritional supplement formulation in said refill supplies.

29. The method according to claim 14, further comprising delivering a first refill supply of said nutraceutical to the patient, said patient being directed according to said regimen to begin taking doses from said refill supply once said initial supply is depleted.

30. The method according to claim 29, wherein said first refill supply is delivered to the patient through delivery services such as the U.S. mail, express mail or overnight courier, and wherein further refill supplies are delivered to the patient monthly through delivery services such as the U.S. mail, express mail and overnight courier as necessary.

31. The method according to claim 30, wherein the patient receives periodic refill supply shipments on a monthly basis.

32. A nutraceutical product containing safe and effective nutritional supplement ingredients for the prevention and/or treatment of cardiovascular disease in a patient, said nutraceutical having a formulation of nutritional supplement ingredients comprising:
folic acid, vitamin B6, vitamin B12, niacin, plant sterols, and omega-3 fatty acids present in appropriate pharmacologically safe and effective proportions and amounts.

33. The nutraceutical product according to claim 32, wherein said nutritional supplement ingredients are provided in one or more delivery vehicles and one or more delivery forms, said vehicles and said forms being packaged together in a single-dose packet or packets.

34. The nutraceutical product according to claim 33, wherein said delivery forms are selected from the group consisting of selected from the group consisting of tablets, capsules, softgels, edible bars, edible wafers, and powders.

35. The nutraceutical product according to claim 32, said nutraceutical further comprising multi-vitamin pills, said multi-vitamin pills being substantially free of vitamin K.

36. The nutraceutical product according to claim 32, said nutraceutical further comprising multi-vitamin pills, said multi-vitamin pills being substantially free of iron.

37. The nutraceutical product according to claim 32, said nutraceutical further comprising multi-vitamin pills, said multi-vitamin pills being substantially free of anti-oxidants.

38. The nutraceutical product according to claim 32, wherein said formulation comprises an amount of folic acid sufficient to provide about 200% of recommended daily intake.

39. The nutraceutical product according to claim 32, wherein said formulation delivers approximately 0.8 mg of folic acid per day.

40. The nutraceutical product according to claim 32, wherein said formulation comprises an amount of vitamin B6 sufficient to provide about 1250% of recommended daily intake.

41. The nutraceutical product according to claim 32, wherein said formulation delivers approximately 25 mg of vitamin B6 per day.

42. The nutraceutical product according to claim 32, wherein said formulation comprises an amount of vitamin B12 sufficient to provide about 16667% of recommended daily intake.

43. The nutraceutical product according to claim 32, wherein said formulation delivers approximately 1 mg of vitamin B12 per day.

44. The nutraceutical product according to claim 32, wherein said folic acid, vitamin B6, and vitamin B12 ingredients are provided along with other non-interfering nutrients in the form of a multi-vitamin.

45. The nutraceutical product according to claim 32, wherein said formulation delivers approximately 500 mg of niacin per day.

46. The nutraceutical product according to claim 45, wherein a daily dosage of niacin is provided in the form of two 250 mg tablets, each tablet being taken at separate times during a day.

47. The nutraceutical product according to claim 32, wherein said formulation delivers approximately 1800 mg of plant sterols per day.

48. The nutraceutical product according to claim 47, wherein said plant sterols are present in a combination of sterol types, said sterol types being selected from the group consisting of beta sistosterol, campesterol, stigmasterol, and brassicasterol.

49. The nutraceutical product according to claim 32, wherein said plant sterols are provided in six softgel capsules with each capsule containing 300 mg of plant sterols, three of said softgel capsules being taken together at two separate times during a day.

50. The nutraceutical product according to claim 49, wherein said two times per day are just before the first meal containing fat, and just before dinner.

51. The nutraceutical product according to claim 32, wherein said omega-3 fatty acids are provided by softgel capsules containing an appropriate amount of natural fish oils.

52. The nutraceutical product according to claim 32, wherein said formulation delivers approximately 1100 mg of said omega-3 fatty acids per day.

53. The nutraceutical product according to claim 32, wherein said omega-3 fatty acids are present in a combination of fatty acid types, said fatty acid types primarily being selected from the group consisting of EPA and DHA.

54. The nutraceutical product according to claim 32, wherein said nutritional supplement ingredients are pre-packaged in single-dose packets where each said packet is adapted to provide one-half of a daily dosage of said formulation, wherein said packets contain supplement pills, said supplement pills comprising:
multi-vitamin pills providing said appropriate amounts of folic acid, vitamin B6 and vitamin B12 according to said formulation;
pills providing said appropriate amount of niacin according to said formulation;
pills providing said appropriate amount of plant sterols according to said formulation; and pills providing said appropriate amount of omega-3 fatty acids according to said formulation.

55. The nutraceutical product according to claim 54, wherein said multi-vitamin pills provide beneficial amounts of other vitamins and minerals but are substantially free of vitamin K and iron.

56. The nutraceutical product according to claim 54, wherein said plant sterols containing pills comprise softgel capsules.

57. The nutraceutical product according to claim 54, wherein said omega-3 fatty acids containing pills comprise softgel capsules of fish oils.

58. The nutraceutical product according to claim 57, wherein said fish oil softgel capsules comprise an effective amount of co-enzyme Q10.

59. The nutraceutical product according to claim 58, wherein said co-enzyme Q10 is present in an amount suitable to deliver a dosage of approximately 50-60 mg daily.

60. A method for treating and/or preventing cardiovascular disease in a patient, said method comprising administering daily to the patient an effective amount of a nutraceutical, said nutraceutical comprising appropriate pharmacologically safe and effective proportions and amounts of folic acid, vitamin B6, vitamin B12, niacin, plant sterols, and omega-3 fatty acids.

61. The method according to claim 60, wherein said nutritional supplement ingredients are pre-packaged in single-dose packets where each said packet is adapted to provide one-half of a daily dosage of said nutraceutical, wherein said packets contain supplement pills, said supplement pills comprising:
multi-vitamin pills providing said appropriate amounts of folic acid, vitamin B6 and vitamin B12;
pills providing said appropriate amount of niacin;
pills providing said appropriate amount of plant sterols; and
pills providing said appropriate amount of omega-3 fatty acids.

62. The method according to claim 61, wherein said multi-vitamin pills provide beneficial amounts of other vitamins and minerals but are substantially free of vitamin K and iron.

63. The method according to claim 61, wherein said plant sterols containing pills comprise softgel capsules.

64. The method according to claim 61, wherein said omega-3 fatty acids containing pills comprise softgel capsules of fish oils.

65. The method according to claim 64, wherein said fish oil softgel capsules comprise an effective amount of co-enzyme Q10.

66. The method according to claim 65, wherein said co-enzyme Q10 is present in an amount suitable to deliver a dosage of approximately 50-60 mg daily.

67. The method according to claim 60, wherein approximately 0.8 mg of folic acid per day is administered.

68. The method according to claim 60, wherein approximately 25 mg of vitamin B6 per day are administered.

69. The method according to claim 60, wherein approximately 1 mg of vitamin B12 per day is administered.

70. The nutraceutical product according to claim 60, wherein said folic acid, vitamin B6, and vitamin B12 ingredients are administered orally along with other non-interfering nutrients in the form of a multi-vitamin.

71. The method according to claim 60, wherein approximately 500 mg of niacin per day are administered.

72. The method according to claim 60, wherein said niacin is administered orally in pill form at two times per day, each said time administering a half-dose of niacin.

73. The method according to claim 60, wherein approximately 1800 mg of plant sterols per day are administered.

74. The method according to claim 60, wherein said plant sterols can be a combination of sterol types, said sterol types being selected from the group consisting of beta sistosterol, campesterol, stigmasterol, and brassicasterol.

75. The method according to claim 60, wherein said plant sterols are administered in half-doses at two separate times during a day.

76. The method according to claim 75, wherein said two times per day are just before the first meal containing fat, and just before dinner.

77. The method according to claim 60, wherein approximately 1100 mg of omega-3 fatty acids per day are administered.

78. The method according to claim 60, wherein said omega-3 fatty acids are present in a combination of fatty acid types, said fatty acid types primarily being selected from the group consisting of EPA and DHA.
